(19) United States
(12) Patent Application Publication
(10) Pub. No.: US 2017/0143344 A1
(43) Pub. Date: May 25, 2017

(54) APPARATUS AND METHOD FOR STAGED COMPRESSION ANASTOMOSIS

(71) Applicant: NOVOGI LTD., Netanya (IL)

(72) Inventors: Leonid MONASSEVITCH, Hadera (IL); Omer CARMEL, Tel- Mond (IL)

(73) Assignee: NOVOGI LTD., Netanya (IL)

(21) Appl. No.: 15/318,775

(22) PCT Filed: Jun. 16, 2015
(86) PCT No.: PCT/IL2015/050608

§ 371 Date: Dec. 14, 2016

Related U.S. Application Data
(60) Provisional application No. 62/012,441, filed on Jun. 16, 2014.

(51) Int. Cl. A61B 17/11

(52) U.S. Cl. CPC A61B 17/1114; A61B 2017/00004

(57) ABSTRACT

A compression assembly for use in compressing tissue comprising a first portion which includes a first compression element and a second portion which comprises a second compression element, at least one support element, at least one spring stopper element, and at least one spring element. Typically the spring stopper element is formed of a bio-degradable or otherwise functionally controllable material. The at least one spring element is in compressive force contact with the second compression element and the tissue to be joined is positioned between the first and second compression elements. A plurality of needles on one of the support elements is operative to pierce the tissue and the first portion of the assembly, holding the first compression element to the second portion of the assembly. The invention is appropriate for joining severed tissue in anastomosis procedures or closing natural or surgically produced tissue perforations.

### BACKGROUND OF THE INVENTION

Excision of a segment of diseased colon or intestine and subsequent anastomosis of the cut end portions is known in the art. Such excision and anastomosis can be carried out by opening the peritoneal cavity or laparoscopically. However, there are significant problems associated with these procedures.

The integrity of the anastomosis must be sound so that there is mimimum risk of the anastomosis rupturing or leaking into the peritoneal cavity. Opening the bowel and exposing the clean peritoneal cavity to contamination increases the risk of postoperative complications.

It is well known that in the rectal region avoidance of dehiscence is difficult. Some patients have a higher risk of postoperative dehiscence, for example, because of certain health problems related to diabetes mellitus, radiation enteritis, generalized peritonitis or treatments such as chemotherapy or treatment with biologic agents. Sometimes the technical factors that ensure good conditions for surgery, such as near-perfect apposition of the two intestinal ends, good vascular supply, lack of tension or lack of distal obstruction, cannot be met.

In these cases, as a rule, a protective diverting stoma is used. The diverting stoma does not prevent leakage but it minimizes the clinical consequence should this complication occur.

The necessity for a later reoperation to re-establish intestinal continuity is the obvious and essential defect of the staged procedure. Therefore an effort to eliminate the protective (temporary) stoma seems to be worthwhile.

It is known that under normal conditions anastomosis has the minimum strength on the 3th -4th postoperative day. Till this time the biological strength of risky anastomosis does not sufficiently increase, but the mechanical strength substantially falls because of inflammatory processes in case of the sutured anastomosis or because of not satisfactory thickness of the necrotic tissue in case of the compression anastomosis.

It would be advantageous to retain the mechanical strength of the anastomosis as long as the biological strength adequately increases.

### BRIEF DESCRIPTION OF THE DRAWINGS

The principles and operation of the system, apparatus, and method according to the present invention may be better understood with reference to the drawings, and the following description, it being understood that these drawings are given for illustrative purposes only and are not meant to be limiting, wherein:

FIGS. 1A-1C show several views of a proximal portion of a CAR assembly, constructed according to an embodiment of the present invention;

FIGS. 2A-2D show several shape-memory alloy stress-strain hysteresis loops produced by the shape-memory elements of a CAR assembly constructed according to an embodiment of the present invention; and

FIG. 3 is a flowchart illustrating a typical treatment flow, according to an embodiment of the present invention.

It will be appreciated that for simplicity and clarity of illustration, elements shown in the drawings have not necessarily been drawn to scale. For example, the dimensions of some of the elements may be exaggerated relative to other elements for clarity. Further, where considered appropriate, reference numerals may be repeated among the drawings to indicate corresponding or analogous elements throughout the serial views.

### DETAILED DESCRIPTION OF THE INVENTION

The following description is presented to enable one of ordinary skill in the art to make and use the invention as provided in the context of a particular application and its requirements. Various modifications to the described embodiments will be apparent to those with skill in the art, and the general principles defined herein may be applied to other embodiments. Therefore, the present invention is not intended to be limited to the particular embodiments shown and described, but is to be accorded the widest scope consistent with the principles and novel features herein disclosed. In other instances, well-known methods, procedures, and components have not been described in detail so as not to obscure the present invention.

The present invention incorporates fully herein U.S. Pat. No. 8,205,782 B2, filed on Jul. 12, 2007, by the same inventor(s), which is a continuation-in-part of U.S. patent application Ser. No. 11/485,604, filed Jul. 12, 2006, now U.S. Pat. No. 7,527,185, issued May 5, 2009, and U.S. Provisional Appl. No. 60/900,723, filed on Feb. 12, 2007.

Mechanical strength of compression anastomosis is determined by the thickness and state of the tissue compressed by an implant. Embodiments of the present invention enable increasing the thickness of the compressed tissue sufficiently by stopping the compression force when an adjusted gap between the ring and the anvil is obtained. This way the time needed to increase the biological strength up to a satisfactory degree is prolonged is enabled.

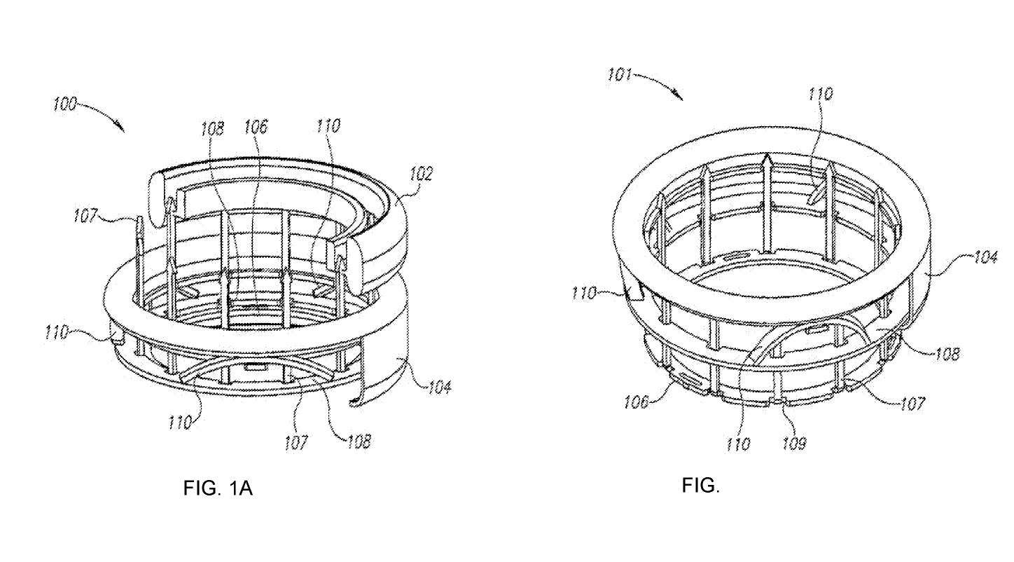

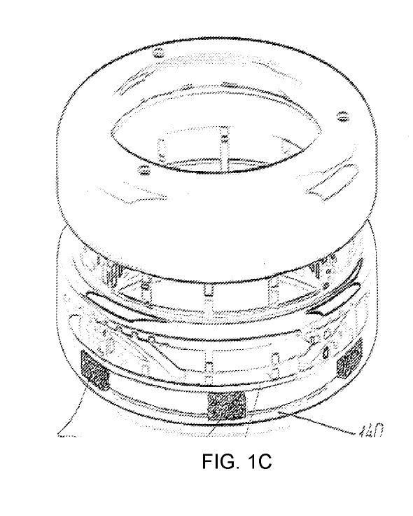

Reference is now made to FIGS. 1A and 1B, which show views of a proximal portion of a CAR assembly constructed according to prior art by the same inventors. FIG. 1B represents a cut away view of only the second portion 101 of CAR assembly 100. The entire CAR assembly 100 is shown in FIG. 1A. CAR assembly 100 includes an anvil disk 102 formed of any of a large number of rigid plastics known to those skilled in the art and a bottom ring 104 which may be formed from any of a large number of plastics or metals known to those skilled in the art. Anvil ring 104 is positioned on the disk's periphery and an anvil inner core 105. Anvil disk 102 may include holes into which the ends of needles 107, to be discussed below, may enter. Alternatively, no such holes need be included and needles 107 themselves pierce and enter plastic anvil disk 102 when a force of sufficient magnitude is exerted on them.

Bottom ring 104 girdles a needle ring 106, CAR flange 108 and one or more spring elements 110. Needle ring 106 includes a plurality of barbed needles 107, each needle 107, typically but without intending to limit other possibilities, spaced substantially equidistant from its two nearest neighbors. Needles 107 are deployed in essentially a circular configuration to conform to the circumference of needle ring 106. Again such a configuration is exemplary only and not intended to be limiting.

Needles 107 may be formed integrally with needle ring 106. Alternatively, they may be joined to needle ring 106 by any of several methods known to those skilled in the art, such as welding, gluing, and pressure fitting. These methods are exemplary only and are not intended to be limiting. The shape of the barbs on the heads of needles 107 as shown in FIGS. 1A and 1B is exemplary only. Any generally penetrating shape may be used as the head of needles 107, even sharp heads without barbs.

CAR flange 108 is typically, but without intending to be limiting, formed from any of a large number of metals or plastics known to those skilled in the art. Needle ring 106 and the plurality of barbed needles 107 are typically, but without intending to be limiting, formed from any of a large number of metals or plastics known to those skilled in the art. In some embodiments the one or more spring elements 110 are constructed from a shape-memory alloy, typically, but again without intending to be limiting, nitinol. Also typically, but without intending to be limiting, spring elements 110, when in their unloaded austenite state, are arch-shaped. The spring elements are positioned to lie on CAR flange 108 between flange 108 and bottom ring 104. The top of the arch contacts the underside that is the closest side, of bottom ring 104. When the shape-memory alloy from which spring elements 110 are formed is in its loaded stress-induced martensite state (or stress-retained martensite state), spring elements 110 lie substantially flat along CAR flange 108 below bottom ring 104. Spring elements 110 are positioned on CAR flange 108 so that their ends can move when going from the spring elements' uncompressed arched shape to the spring elements' flat compressed shape and vice versa.

Needle ring 106 is positioned below CAR flange 108. CAR flange 108 has indentations 109 along its inner generally circular circumference through which barbed needles 107 extend from needle ring 106 past CAR flange 108.

Spring elements 110 have been described herein as having an arched uncompressed configuration when not compressed and a flat configuration when compressed; these are essentially leaf springs. The present invention also contemplates other possible spring forms and configurations, including conventional coiled configurations.

In what has been described herein throughout, CAR assembly 100 has been described as having a separate CAR flange 108 and a needle ring 106. In other embodiments, there may be only a single element, essentially the needle ring with needles 107 affixed thereon. The CAR flange may be eliminated. In such an embodiment, spring elements 110 are positioned on the needle ring and they contact the bottom of bottom ring 104. The spring elements are movable on needle ring 106 and they are capable of moving from their compressed to uncompressed configurations/shapes and vice versa. In this latter embodiment, spring elements 110 are typically, but without intending to be limiting, deployed in their non-compressed austenitic state. When a CAR flange 108 is employed the spring elements 110 are typically deployed in their compressed martensitic state.

It should be noted that all ring or ring-shaped elements discussed herein, including the claims, with respect to the CAR assembly 100, contemplate, in addition to the use of circular-shaped elements, the possibility of using elliptical, oval or other shaped elements. The use of “ring” should not be deemed as shape limiting for the rings elements described and illustrated hereinabove. These ring elements include, but are not limited to, the needle ring 106, the CAR flange 108, and the bottom ring 104.

It should also be noted that the use of the term “bottom ring” as a term for element 104 should not be deemed as denoting anything about the specific spatial and functional relationship between this element and the other elements of the CAR assembly 100. The spatial and functional relationship of element 104 and the other elements of assembly 100 are defined by the description and the drawings.

As can be seen in FIG. 1C, the assembly may include one or more mechanical stopper element(s) 130, which may be configured to provide a fixed gap of selected distance between the anvil ring and the anvil disk, when in locked configuration. In some embodiments stopper 130 may be biodegradable and thereby designed to break down and allow the disk and ring to continue to compress the tissue, thus accomplishing the creation of the anastomosis. In one example, stopper 130 may be constructed from porous magnesium in the form of a circular spacer. This spacer is generally located between the metal ring floor 140 and the needle support flange 135. In the adjusted time (e.g., several days), the stopper may corrode and break down, allowing the disk and ring to be freed to be freed from the stopper caused gap, to continue with the anastomosis process. In further embodiments the stopper may be externally or otherwise deployed at a selected time to optimally allow for anastomosis creation.

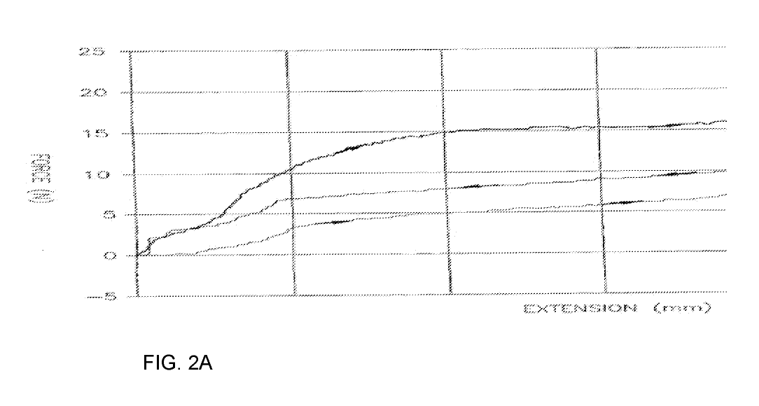

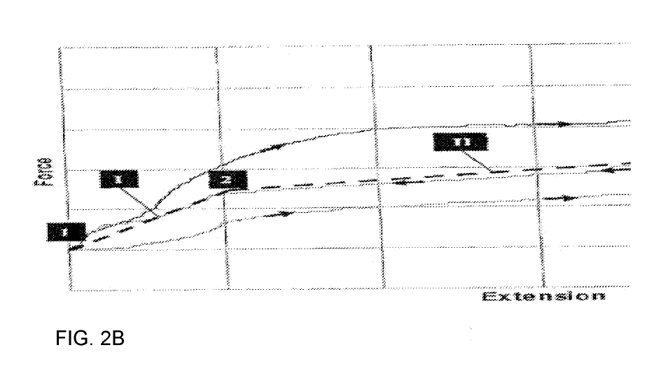

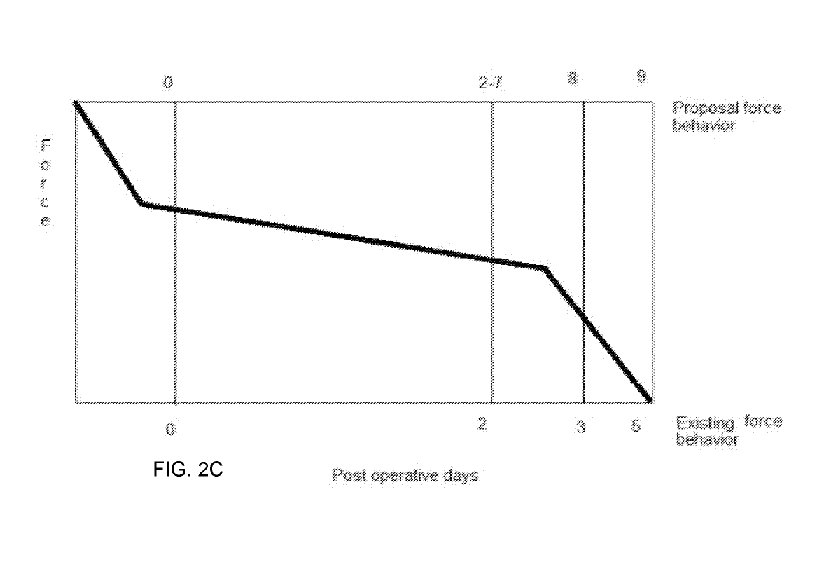

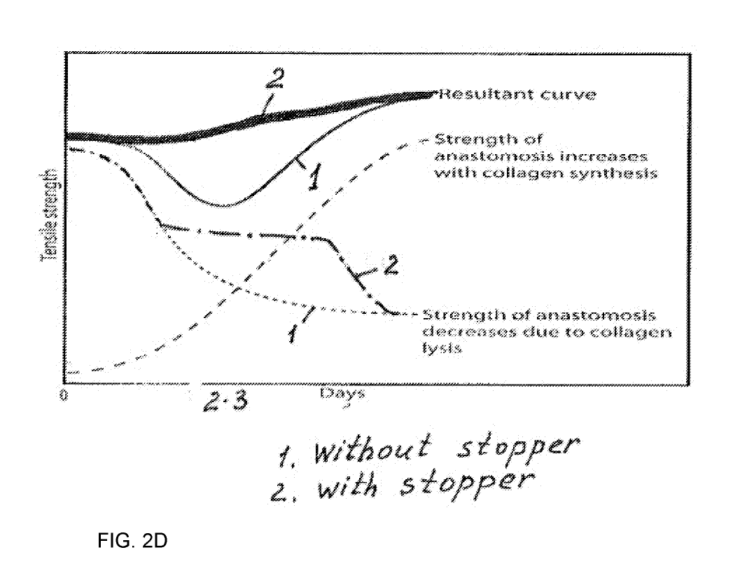

Reference is now made to FIGS. 2A and 2B, which indicate examples of prior art force-extension-compression hysteresis loops for the CAR assembly having spring elements formed of a shape-memory (SM) alloy, by the same inventors. FIGS. 2A and 2B are essentially identical except for the additional annotation in FIG. 2B required for the discussion below.

As noted above, in some embodiments, the spring elements 110 of the CAR assembly of the present invention may be constructed of a shape-memory alloy, typically but without intending to be limiting, Ni—Ti alloy. They make use of the substantially “plateau-like” region in curve B of the hysteresis loops shown. Curve B represents the removal of the shape-changing stress from spring elements110. “Plateau-like” region II in curve B (between points 2 and 3 in FIG. 2B) indicates that a slowly decreasing force is exerted on the tissue for which anastomosis is being effected over a defined extension range. While the rate of change of force (F) with respect to extension (x), i.e. dF/dx, in the “plateau-like” region II, is not truly zero, it is significantly smaller than the rate of change in the other regions (regions I and III) of curve B in FIG. 2B. Alternatively, the situation in FIGS. 2A and 2B can be discussed in terms of the material's Young's modulus as follows. The Young's modulus of the material in the “plateau-like” region (region II in FIG. 2B between points 2 and 3 in the curve shown there) is less than in at least one of the regions (I and III) of the graph adjacent to the “plateau” region. The “plateau” region, typically, extends over the greater part of the spring element's extension range contemplated to be used in a compression procedure. The broken line in FIG. 2B allows for better visualization of the changes in the slope of the force-extension curves.

The present invention has been described above as using stress-induced shape changes in spring elements 110. The hysteresis loop for such a situation is represented by curves A-B. The present invention also contemplates using shape changes induced by cooling and stress. A hysteresis loop, shown as curves C-B and having a similar “plateau” region in curve B, reflects the situation when such conditions are employed. Arrows on the hysteresis loops of FIGS. 2A and 24B show the direction in which the stress is applied and removed under each method of martensitic transformation.

It will be appreciated by persons skilled in the art, that in general, a compression assembly employing spring elements 110 constructed of a shape memory (SM) alloy may be used in one of two ways. The alloy may be deformed at room temperature in its austenite state thus transforming it into its martensite state, often known as stress-induced martensite (SIM) (curve A). This employs the alloy's superelastic behavior. While in its SIM state, the spring's SM alloy is restrained in its deformed shape by a restraining means. After positioning the compression assembly in the body and increasing the spring element's temperature to body temperature and removing the restraining means, the alloy returns to its austenite state and the spring to its original shape along a path represented by curve B. As the spring returns to its uncompressed configuration, it presses on the tissue with a relatively slowly decreasing force, i.e. small dF/dx ratio, over the greater part of its extension range thereby bringing about anastomosis.

In the second way of using a spring constructed from a shape memory (SM) alloy, the superelastic plasticity behavior of the alloy is employed. The alloy of the spring is first cooled transforming the alloy, at least partially, into its martensite state (curve C). The alloy is then deformed, i.e. the spring is then loaded, and retained using a special restraining means in its deformed martensite state. This martensite state is often referred to as the stress-retained martensite (SRM) state. The alloy/spring is then warmed to body temperature. When the spring, in the present invention spring elements 110, is released from the restraining means at body temperature, the alloy returns to its austenite state, and the spring returns to its original uncompressed shape (via curve B). As the spring returns to its original configuration, it presses on the tissue with a relatively slowly decreasing force, i.e. small dF/dx ratio, over the greater part of its extension range thereby bringing about anastomosis.

It should be noted that in both cases, the return to the austenite uncompressed, unloaded state from the compressed, loaded martensite state is along the same path, curve B. In both cases, the same slowly decreasing force, i.e. small dF/dx ratio, represented by the “plateau-like” region of curve B, is recovered.

FIGS. 2A and 2B show that a slowly decreasing force (“plateau-like” region II in curve B) may be used to bring about anastomosis. In known prior art, on the other hand, any spring element used is constructed of regular, non-shape memory materials. Therefore, the force applied by these spring elements is in direct relation to displacement i.e. Hooke's law. Additionally, the maximum reversible strain of spring elements made from conventional metals is on the order of about 0.3%.

In view of the direct relationship to displacement in conventional spring materials, the compressive force to effect anastomosis is a function of tissue thickness. Additionally, in view of the small reversible strain, a large “gap”, that is distance between the first and second portions of the CAR assembly, would be required to provide the necessary compressive force.

As already noted, the first factor, in effect, makes the anastomosis process using prior art devices a function of tissue thickness. However, in order to enhance the anastomosis with a strong seal at the join, approximately the same force should be applied throughout the process, and the force should be essentially the same irrespective of tissue thickness. It should be noted that too much force may lead to premature detachment of the CAR assembly, possibly even before healthy new scar tissue is formed. Too little force may result in the CAR assembly detaching only after a very long time. Additionally, it may not produce ischemia. Spring elements formed from shape-memory alloys, as in the present invention, provide a relatively slowly decreasing force independent of tissue thickness, in their “plateau-like” region without premature or excessively long detachment times. These elements may also produce ischemia, as required.

As also noted above, the small reversible strain of regular spring materials may require an increased size for the CAR assembly. The resulting increase in assembly size would inter alia impair the assembly's expulsion from the bowel after anastomosis has been completed.

The use of a shape memory alloy, typically nitinol, for forming a spring element, as in some embodiments of the present invention, allows for the use of a thin nitinol leaf as a spring element. The leaf, typically, but without intending to be limiting, may have a thickness of about 0.5 mm. When the leaf deforms, the CAR “gap”, the distance between the first and second portions of the CAR assembly, increases. What is herein described as being a small leaf spring allows for the use of nitinol's relatively large reversible strain (˜6%) as contrasted with a conventional metal's small reversible strain (˜0.3%). With conventional spring metals similar deformations can not be achieved; a physically larger spring such as a coil spring must be used. This would lead to larger assembly sizes.

It therefore was realized by the inventors that in some embodiments, a resilient element, here at least one spring element, formed from a shape memory material, such as nitinol, would maximize the efficiency of the element in speeding healing. In effect, use of shape memory materials allows for maximizing healing by taking into consideration the needs of the healing and necrotic processes as tissue thickness decreases during the processes.

At the far end of the X axis on the force-extension curves of FIGS. 2A and 2B, i.e. at the beginning of the compression/healing/necrotic process where tissue thickness (X) is greatest, nitinol elements may allow for faster hemostasis by providing their greatest force in region III shown in FIG. 2B. As is generally known, greater pressure assists in hemostasis.

As healing continues and tissue thickness is reduced a relatively slowly decreasing force, regardless of tissue thickness (X), is more beneficial (region II shown in FIG. 2B). This is more advantageous because a slowly decreasing force, as thickness (X) decreases, allows for a better seal between the tissues being compressed and joined. There is therefore less chance for leakage and sepsis.

A usable figure of merit for determining the suitability of a material in forming the resilient elements, here spring elements, required in constructing the compression assemblies would be Fb/Fa2, where F is the force generated by the spring element constructed of the given material at the high force end (point b) of region II and the low force end (point a) of region II (“plateau-like” region). In FIG. 2B, the high force end of region II is represented by the force at point3 and the low force end of region II by point 2.

Finally, at the end stage of the healing process, i.e. the necrotic phase, where tissue thickness X is smallest, a relatively controlled detachment of the compression assembly is required. The force drops to zero as tissue thickness (X) drops to zero (region I of FIG. 2B). This prevents the compression assembly from detaching before necrosis is complete. The compression assembly would tear through the thin tissue if the force did not decrease to zero, and detachment would otherwise occur before healing was complete.

It should be understood that embodiments of the present invention also contemplate other materials which do not behave according to Hooke's law and which provide a relatively slowly decreasing force over a substantial portion of the spring element's expected range of extension as in FIGS. 2A and 2B. Therefore, dF/dx should be small over a substantial portion of the expected extension range; alternatively the Young's modulus of the material should be smaller over a substantial portion of the expected extension range than the adjacent portions of the graph.

It will be understood by a person skilled in the art that all materials having characteristics similar to those discussed above for Ni—Ti alloys and spring elements made from such alloys, may be used to form the resilient elements, here the spring elements, used in compression assemblies constructed according to the present invention. The use and discussion above of shape memory materials is not intended to limit the choice of materials that may be used for such resilient elements.

It will be appreciated by persons skilled in the art that there is a direct relationship between the size and thickness of the CAR assembly 100 and applicator 10 used in the surgical procedure disclosed above and the size, shape and type of organ to be treated. A CAR assembly 100 of a particular size is selected so as to achieve an aperture of a requisite size as appropriate to the situation and the hollow organ to be subjected to anastomosis. Clearly, a smaller size is appropriate for use in the upper bowel and a larger size in the lower bowel.

It should also be understood that the present invention also contemplates a case where spring elements 110 may be deployed in their unloaded, uncompressed, here arched, configuration. In such a configuration, the alloy from which the spring elements are formed is initially in its austenite state. After the second portion 101 of CAR assembly 100 s deployed (with its spring elements 110 in their unloaded austenite state) on the distal end of CAR applicator 10, a load can be applied to CAR flange 108. Such a load can be applied by a load lip, load teeth or load protrusions. After bringing spring elements 110 to their loaded martensite state, anvil disk 102 of the CAR assembly 100 is brought towards the second portion 101 of CAR assembly 100 with tissue to be anastomosized held therebetween. When the tissue is held sufficiently securely by anvil disk 102 and second portion 101, spring elements 110 are unloaded and they begin to arch causing bottom ring 104 of CAR assembly 100 to compress the tissue held against anvil disk 102 and anastomosis can occur. In this embodiment, as in prior embodiments, spring elements 110 may be positioned on CAR flange 108 and in contact with bottom ring 104. Alternatively, when no CAR flange is present spring elements 110 may be positioned on needle ring 106 so that it is in contact with bottom ring 104.

As noted previously, all ring or ring-shaped elements discussed herein with respect to the CAR assembly 100, contemplate, in addition to the use of circular-shaped elements, the possibility of using elliptical, ovoid or other shaped elements. The use of “ring” should not be deemed as shape limiting for the ring elements described and illustrated hereinabove. These ring elements include, but are not limited to, the needle ring, the CAR flange, and the bottom ring. Among the other shapes contemplated for use with elements of the present invention are hexagonal, octagonal and other closed curve shapes. Additionally, substantially linear elements may also be used. Assemblies including linear elements are not necessarily contemplated for use in anastomosis procedures but may be used in compression closure of resections, excisions, perforations and the like.

It should also be borne in mind that the applicator discussed herein with assembly 100 is only exemplary and not intended to be limiting. Other applicators may also be designed by persons skilled in the art that may be used with CAR assembly 100.

As can be seen with reference to FIG. 2C, the timeline for the force behavior of the assembly may be lengthened substantially with the usage of the fixed gap element or stoppers, according to some embodiments. As can be seen, the force behavior post operationally may be selectively sustained for longer periods of time, such as for 2-7 days as shown, by using a stopper that may be dissolved or otherwise disabled around 2-7 days, after which the force of the Nitinol spring element will take over in accordance with its pre-programmed configuration, from days 7-9. Of course, other intervals, combinations of intervals may be used, and other materials or mechanisms may be used, as needed.

Reference is now made to FIG. 2D, which is an example of a graph showing tensile strength of an anastomosis over time, according some embodiments. As can be seen, the strength of the Anastomosis may be maintained high by usage of the stopper, immediately following a procedure. As a result, the initial thickness of compressed tissues and correspondingly the larger volume of collagen are substantially maintained in their initial merging position. In this way, the resultant force curve increases during the critical first days, preventing the anastomosis dehiscence. The mechanical support of anastomosis strength during this initial period increases the time needed for collagen synthesis. Following this initial period the stopper is disengaged and therefore the spring mechanism may be engaged. The activation of the spring mechanism thereby allows the Ring to be expelled at the time when the anastomosis is quite strong, at the expense of a synthesized collagen.

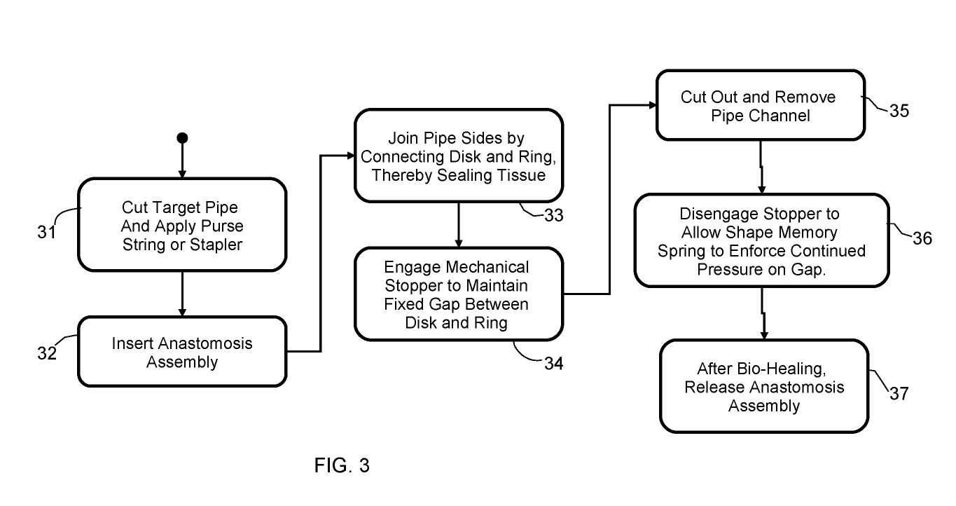

FIG. 3 schematically illustrates a series of operations or processes that may be implemented to implement an anastomosis procedure, according to some embodiments of the present invention. As can be seen in FIG. 3, at step 31 a typical purse string or stapler procedure is used to close up a pipe substantially on its sides, to allow place for inserting of the anastomosis assembly. At step 32 the anastomosis assembly is inserted on the sides of the pipe to be closed. At step 33 the sides may be closed by joining together the anvil disk and ring, for example, using spikes to mechanically connect the ring and anvil, with the pipe tissue being seal between the disk and ring. At step 34, the stopper is mechanically engaged to maintain a fixed gap between the disk and ring, at a selected initial gap depth designed to allow the initially sealed tissue to maintain integrity as initial healing occurs. At step 35, a channel is cut out in the pipe, in the inside of the anastomosis assembly, and the inside tissue is removed to open the channel. At step 36, once healing happens and/or when stopper biodegrades or otherwise disengages, the fixed gap established at step 34 is automatically released. At step 37 the spring action of the spring mechanism is engaged, to ensure continued compression pressure on the tissue being sealed between the disk and ring, preferably at a pressure of force configured to provide optimal bio healing for a selected time period. At step 38, when bio healing has substantially been achieved, ring and disk elements are automatically released and released through the pipe. Any combination of the above steps may be implemented. Further, other steps or series of steps may be used.

In some embodiments, where the spring mechanism is at least partially constructed from a shaped memory material, the shaped memory spring mechanism may be engaged to controllable control the amount of compression pressure applied to the healing area between the disk and the ring.

The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

What is claimed is:

1. A compression coalescence assembly which comprises:
a first portion which includes a first compression element; and
a second portion which includes a second compression element positioned substantially parallel to and spaced apart from said first compression element, said first and second compression elements being adapted to be brought together by an attachment mechanism;
at least one support element for supporting said attachment mechanism;
at least one spring element for providing a restorative force, said element behaving in a manner other than that predicted by Hooke's Law over at least a portion of its expected extension range, said spring element positioned on one of said at least one support elements and in compressive force transmissive contact with said second compression element; and
at least one stopper element which is designed to provide a fixed gap between said first portion and said second portion, for a selected time interval.

2. A compression coalescence assembly according to claim 1 wherein said at least one stopper element is constructed from a biodegradable material.

3. A compression coalescence assembly according to claim 2 wherein said material is constructed at least partially from a shape memory alloy.

4. A compression coalescence assembly according to claim 2 wherein said material is degradable by corrosion alloys.

5. A compression coalescence assembly according to claim 3 wherein said alloys is magnesium.

6. A compression coalescence assembly according to claim 4 wherein said magnesium has a porous structure.

7. A compression coalescence assembly according to claim 2 wherein said material is a biodegradable polymer.

8. A compression coalescence assembly according to claim 2 wherein said material is a dissoluble material.

9. A compression coalescence assembly according to claim 1 wherein said at least one stopper element is a permanent element.

10. A compression coalescence assembly according to claim 1 wherein said at least one stopper element provide a gap of 0.5-2.0 mm in width.

11. A compression coalescence assembly according to claim 1, which is either an anastomosis device or clip.

12. A compression assembly as in claim 1, wherein said at least one spring element has a force-extension graph composed of a first region, a second region and an intermediate region lying between said first and second regions, and where in said intermediate region, the force-extension slope is substantially higher than the force-extension slope of at least one of the two adjacent regions.

13. A compression assembly of claim 1, wherein said at least one spring element is at least partially formed from a material which has a recoverable strain of at least about 4%.

14. A compression assembly of claim 1, wherein said first and second compression elements and said at least one support element are formed having the same shape, the shape selected from the group consisting of circular, elliptical, oval and linear shapes.

15. An assembly as in claim 1, wherein said at least one spring element is brought to its compressed configuration, and the material from which it is formed to its martensitic state, by applying thereto a compressive stress.

16. A method for providing compression anastomosis, comprising:
initiating a phase of approximation of the superficial layers of the organs which are intended to be coalescenced;
initiating a phase of detention of organs in the approximated position, using a mechanical stopper element in a compression anastomosis apparatus, for a selected time interval; and
automatically initiating a phase of compression of the approximated organs, using a spring element in a compression anastomosis apparatus, for a selected time interval.

17. The method of claim 16, wherein said approximation includes:
positioning the tissue to be compressed between first and second portions of a compression assembly operable for compressing tissue;
moving the first portion of the assembly into close proximity to the second portion so as to hold the tissue therebetween;
compressing the tissue held between the first and second portions of the compression assembly with a force produced by at least one spring element which provides a non-Hooke's Law restorative force; and
wherein the at least one spring element exhibits a force-extension graph composed of a first region, a second region and an intermediate region lying between the first and second regions, and wherein the intermediate region the force-extension slope is substantially less than the force-extension slope of at least one of the two adjacent regions.

18. The method of claim 16, wherein said spring element is at least partially formed from a material that is a shape memory material.

19. The method of claim 17, further comprising the step of cooling the at least one spring element so that the shape memory material is brought to its martensitic state.

20. The method of claim 16, further including one or more of the deploying the at least one spring element when in its compressed configuration, the material from which it is formed being in its martensitic state, and deploying the at least one spring element in its non-compressed configuration, the material from which the at least one spring element is at least partly formed being in its austenite state.
